(19) United States
(12) Patent Application Publication
(10) Pub. No.: US 2014/0377171 A1
(43) Pub. Date: Dec. 25, 2014

(54) Imaging and Treatment of Neuroendocrine Tumors with Glucose - Dependent Insulinotropic Polypeptide or Analogues or Antagonists Thereof

(71) Applicant: Jean Claude Reubi, Bern (CH)

(72) Inventor: Jean Claude Reubi, Bern (CH)

(21) Appl. No.: 14/124,279

(22) PCT Filed: Jun. 8, 2012
(86) PCT No.: PCT/EP2012/060944

§ 371 Date: Aug. 15, 2014

(30) Foreign Application Priority Data
Jun. 10, 2011 (EP) 11169574

(51) Int. Cl. A61K 51/08; A61K 38/26; A61K 38/22

(52) U.S. Cl. CPC A61K 51/088; A61K 38/22; A61K 38/26
USPC 424/1.45; 530/300

(57) ABSTRACT

The invention relates to a method of imaging pancreatic β-cells, endocrine gastroenteropancreatic tumors and bronchial and thyroid neuroendocrine tumors and a method of treating endocrine gastroenteropancreatic tumors and bronchial and thyroid neuroendocrine tumors by targeting of glucose-independent insulinotropic polypeptide receptors (GIP receptors). Compounds considered are GIP or a GIP analog, each carrying a radionuclide, optionally complexed through a chelator. Non-radioactive GIP receptor antagonists as such are also considered in the long-term treatment of the mentioned tumors. The invention also relates to the use of a combination of GIP or a GIP analog, each carrying a radionuclide, with a GLP-1 agonist and/or somatostatin analogs, also carrying a radionuclide.

### FIELD OF THE INVENTION

The invention relates to a method of imaging pancreatic β-cells, endocrine gastroenteropancreatic tumors and bronchial and thyroid neuroendocrine tumors and a method of treating endocrine gastroenteropancreatic tumors and bronchial and thyroid neuroendocrine tumors by targeting of glucose-independent insulinotropic polypeptide receptors (GIP receptors).

### BACKGROUND ART

Incretins such as glucagon-like peptide-1 (GLP-1) or glucose-dependent insulinotropic polypeptide (GIP) are important glucose-dependent insulin secretagogues released primarily from the gastrointestinal tract in response to nutrient intake (Hoist J J, Physiological Reviews 2007; 87:1409-1439; Hoist J J, Vilsboll T and Deacon C F, Mol Cell Endocrinol 2009; 297:127-36). In addition to their regulation of glucose-dependent insulin secretion, those peptides have other common actions on β-cells, including stimulation of cell proliferation and reduction of β-cell apoptosis. The concordant incretin effects of GLP-1, including stimulation of insulin, suppression of glucagon, delaying gastric emptying and increasing β-cell mass, have suggested its possible use for diabetes 2 treatment. Therefore, stable synthetic GLP-1 analogs such as exenatide, liraglutide or taspoglutide have been designed and developed for that indication (Ahren B, Nature Reviews Drug Discovery 2009; 8:369-385; Estall J L and Drucker D J, Current Pharmaceutical Design 2006; 12:1731-1750; Knop F K, Vilsboll T and Hoist J J; Current Protein and Peptide Science 2009; 10:46-55; Sebokova E, Christ A D, Wang H, Sewing S, Dong J Z, Taylor J, Cawthorne M A and Culler M D, Endocrinology 2010; 151:2474-2482).

Up to now, however, only very little is known of another peptide receptor belonging to the incretin family, the glucose-independent insulinotropic polypeptide (GIP) receptor, and its expression in human tumors. Furthermore, current knowledge of the GIP receptor expression in normal human tissues is very incomplete as well. It is based largely on receptor mRNA investigations in whole organ preparations, but only little information is available on the receptor protein expression, the exact tissue localization of the receptor, and receptor density levels in human organs.

### SUMMARY OF THE INVENTION

The invention relates to a method of imaging pancreatic β-cells, endocrine gastroenteropancreatic tumors and bronchial and thyroid neuroendocrine tumors comprising administering glucose-independent insulinotropic polypeptide (GIP) or a GIP analog, each carrying a radionuclide, optionally complexed through a chelator, or other substituent useful for imaging. Likewise the invention relates to glucose-independent insulinotropic polypeptide (GIP) or a GIP analog, each carrying a radionuclide, optionally complexed through a chelator, or other imaging substituent, for use in imaging pancreatic β-cells, endocrine gastroenteropancreatic tumors and bronchial and thyroid neuroendocrine tumors.

Furthermore the invention relates to a method of treating endocrine gastroentero-pancreatic tumors and bronchial and thyroid neuroendocrine tumors comprising administering a therapeutically effective amount of glucose-independent insulinotropic polypeptide (GIP) or a GIP analog, each carrying a radionuclide, optionally complexed through a chelator, or other substituent useful for tumor treatment, to a patient in need thereof. Likewise the invention relates to glucose-independent insulinotropic polypeptide (GIP) or a GIP analog, each carrying a radionuclide, optionally complexed through a chelator, or other substituent useful for tumor treatment, for use in treating endocrine gastroenteropancreatic tumors and bronchial and thyroid neuroendocrine tumors.

Furthermore the invention relates to a method of treating endocrine gastroentero-pancreatic tumors and bronchial and thyroid neuroendocrine tumors comprising administering a therapeutically effective amount of a glucose-independent insulinotropic polypeptide (GIP) analog, preferably a glucose-independent insulinotropic polypeptide receptor (GIP-R) antagonist, to a patient in need thereof. Likewise the invention relates to a GIP analog for use in treating endocrine gastroenteropancreatic tumors and bronchial and thyroid neuroendocrine tumors.

The invention further relates to a combination of GIP or a GIP analog, each carrying a radionuclide, optionally complexed through a chelator, or other substituent useful for tumor treatment, together with a GLP-1 agonist and/or somatostatin analog, each carrying a radionuclide, for use in the treatment of gastroenteropancreatic tumors and bronchial and thyroid neuroendocrine tumors; and to a method of treatment comprising administering a therapeutically effective amount of such a combination.

### BRIEF DESCRIPTION OF THE FIGURES

FIG. 1: GIP-R in a pancreatic NET (neuroendocrine tumor, A-C) and an ileal NET (D-F).
A: Section with insulin immunohistochemical staining. Bar=1 mm. Arrows=pancreatic islets; Tu=tumor.
B: Autoradiogram showing total binding of ¹²⁵I-GIP.
C: Autoradiogram showing binding of ¹²⁵I-GIP in presence of 10⁻⁶ M GIP (nonspecific binding).
D: Hematoxylin-eosin stained section. Bar=1 mm. m=mucosa, Tu=tumor.
E: Autoradiogram showing total binding of ¹²⁵I-GIP.
F: Autoradiogram showing nonspecific binding.

Both tumors are strongly expressing GIP receptors. Islets in adjacent pancreas in A are also GIP receptor-positive (B) while ileal mucosa in D is receptor-negative (E).

FIG. 2: GIP receptor positive, but somatostatin receptor or GLP-1 receptor-negative insulinoma.

Upper row: A GIP receptor-positive but somatostatin receptor-negative benign insulinoma.

Lower row: A GIP receptor-positive but GLP-1 receptor-negative malignant insulinoma.
A, F: Hematoxylin-eosin stained sections. Bars=1 mm.
B, G: Autoradiograms showing total binding of ¹²⁵I-GIP.
C, H: Autoradiograms showing nonspecific binding of ¹²⁵I-GIP.
D: Autoradiogram showing total binding of ¹²⁵I-Tyr³-Octreotide.
E: Autoradiogram showing nonspecific binding.
I: Autoradiogram showing total binding of ¹²⁵I-GLP-1.
J: Autoradiogram showing nonspecific binding.

FIG. 3: GLP-1 receptors in normal human pancreas.
A: Insulin immunohistochemistry. Arrows=islets. Bar=1 mm.
B: Autoradiogram showing total binding of ¹²⁵I-GIP.
C: Autoradiogram showing nonspecific binding.

FIG. 4: Competition experiments in a pancreatic NET (vipoma) (A) and in normal pancreatic islets (B). Human GIP(1-30) displaces with high affinity the ¹²⁵I-GIP radioligand, while GLP-1 displaces it with low affinity.

Log[c] (M): Log concentration of compound in mol

¹²⁵I GIP 1-30 (h) s.b. %: Specific binding (%) of human ¹²⁵I-GIP 1-30

FIG. 5: GIP stimulates calcitonin release in TT cells.

Dose response effects of GIP on calcitonin secretion: TT cells (human medullary thyroid cancer cells) were incubated for 48 hours in challenge medium (RPMI 1640 without FCS) and subsequently stimulated with 1 nM, 10 nM, 100 nM and 1000 nM GIP for 3 hours.

Calcitonin levels (C, pg/ml) were determined from the cell supernatant using the Calcitonin-kit from Siemens.

### DETAILED DESCRIPTION OF THE INVENTION

An overexpression of the glucose-independent insulinotropic polypeptide (GIP) receptor in tumors in vitro was found. The invention relates to a method of targeting tumors in vivo based on this observation. Moreover the invention relates to a method of targeting tumors with multiple incretin peptide analogs directed against different incretin peptide receptors (including the GIP receptor) expressed in the same tumor. Such a method will be more efficient than present treatment schedules using just one kind of peptide or peptide analog.

The incretins, glucagon-like peptide 1 (GLP-1) and glucose-dependent insulinotropic polypeptide (GIP), are related hormones secreted from the gastrointestinal mucosa in response to nutrient entry. They play a major role in glucose homeostasis by stimulation of insulin secretion. Both GIP and GLP-1 exert their effects through interaction with structurally related G-protein coupled receptors, which exhibit considerable amino acid homology and utilize overlapping signal transduction pathways in beta cells of pancreatic islets. Several incretin analogs, specifically GLP-1 analogs, have already been developed for diabetes therapy, while GIP analogs are still in an earlier stage of development but have the potential to be implicated in therapy in various indications.

Up to now, only very little is known about the GIP receptor expression in human tumors (McIntosh C H, Widenmaier S and Kim S J, Vitam Horm 2009; 80:409-71). Furthermore, current knowledge of the GIP receptor expression in normal human tissues is very incomplete as well.

For the purpose of the present invention GIP receptor protein expression was assessed, firstly, in a broad spectrum of human tumors, predominantly gastrointestinal, bronchial and thyroid tumors, and, secondly, in normal human tissues considered common sites of tumor origin and metastasis, using in vitro receptor autoradiography. This method has several advantages over other techniques: It identifies receptor binding sites which represent the in vivo target structures, it allows assessing the binding affinity of the receptor, and it correlates with morphology and permits quantification of the receptor density (Reubi J C, Endocr Rev 2003; 24:389-427). Moreover, the results of GIP receptor expression can be compared in the same tumors directly with the status of other peptide receptors, such as the somatostatin receptor and/or GLP-1 receptor, two receptors prominently expressed in endocrine gastroenteropancreatic tumors (Reubi J C and Waser B, Eur J Nucl Med 2003; 30:781-793) and characterized by important clinical implications.

Peptide or cDNA sequencing has revealed that the GIP molecule is a 42 amino acid polypeptide (GIP₁₋₄₂) in all species. Structure-activity studies on GIP and GIP analogs have identified the N-terminus and central region of the GIP molecules as being critical for biological activity. Truncated forms of GIP, including GIP₁₋₃₉ and GIP₁₋₃₀ (Wheeler M B, Gelling R W, McIntosh C H et al, Endocrinology 1995; 136:4629-4639) retain a high degree of biological activity. However, fairly modest changes to Tyr¹-Ala² at the N-terminus can drastically reduce bioactivity. Cleavage by Dipeptidyl peptidase IV (DPP-IV) results in a peptide (GIP₃₋₄₂) lacking insulinotropic activity. The high affinity binding region of GIP₃₋₄₂ resides within the region Phe₆-Lys₃₀ (Gelling R W, Reg Peptides 1997; 69(3):151-154). GIP₆₋₃₀and GIP₇₋₃₀-NH₂bind to the receptor with high affinity, but act as antagonists (U.S. Pat. No. 7,091,183). GIP₃₋₄₂can also act as an antagonist of GIP₁₋₄₂ induced cAMP production in vitro. Both GIP₁₋₁₄and GIP₁₉₋₃₀ are capable of receptor binding and activation of adenylyl cyclase and joining the two peptides with linkers that enhance helix formation in the C-terminal (19-30) portion of GIP produces peptides with enhanced in vitro activity (Manhart S, Hinke S A, Mclntosch C H, Pederson R A, Demuth H U, Biochemistry 2003; 42:3081-3088).

The human GIP receptor gene is located on chromosome 19q13.3 (Gremlich S, Porret A, Hani E H, Cherif D, Vionnet N, Froguel P, Thorens B, Diabetes 1995; 44:1202-1208) and contains 14 exons and 12 introns, with a protein coding region of 12.5 kb. The pancreatic GIP receptor is a glycoprotein that was originally identified in insulinoma cell extracts. Cross-linking studies provided an estimated molecular weight of ˜59 kDa. GIP receptors cDNAs were subsequently cloned from a number of different species. The GIP receptor belongs to the secretin B-family of the seven transmembrane G-protein-coupled receptor (GPCR) family that includes, among others, the receptors for secretin, glucagon, GLP-1, GLP-2, VIP, GRH, and PACAP (Mayo K E, Miller L J, Bataille D et al, Pharmacol Rev 2003; 55:167-94). Receptor expression studies in primary cell lines have facilitated detailed analysis of the regions responsible for ligand binding. The amino terminal domain (NT) contains consensus sequences for N-glycosylation, supporting the proposal that it is a glycoprotein (Amiranoff B, Vauclin-Jacques N, Laburthe M, Life Sci 1985; 36:807-813). Chimeric GIP-GLP-1 receptor studies demonstrated that the NT of the GIP receptor constitutes a major part of the ligand-binding domain, and the first transmembrane (TM) domain is important for receptor activation. GIP binds in an α-helical conformation, with the C-terminal region binding in a surface groove of the receptor, largely through hydrophobic interactions. The N-terminus of GIP remains free to interact with other parts of the receptor. Site-directed mutagenesis studies showed that the majority of the GIP receptor carboxy-terminal tail (CT) is not required for signaling, a minimum chain length is required for expression, and sequences within the CT play specific roles in adenylate cyclase coupling.

TABLE 1
List of GIP analogs
The listed compounds are all published GIP analogs being suitable to coupling with
chelators and radionuclide and therefore adequate for GIP receptor imaging.

| | |
|---|---|
| Agonists | |
| N-Ac GIP (Lys³⁷ PAL) | Irwin G, Peptides 2006; 27(4): 893-900 |
| D-Ala²-GIP | Lamont BJ, Diabetes 2008; 57(1): 190-198 |
| D-Ala²-GIP(1-30) | Widenmaier SB, PLoS ONE 2010; 5(3): e9590 |
| GIP (Lys¹⁶ PAL) | Irwin N, J Med Chem 2006; 49(3): 1047-1054 |
| GIP (Lys³⁷ PAL) | ″ |
| N-Ac GIP | Cassidy RS, Biol. Chem 2008; 389(2): 189-193 |
| N-Ac GIP (Lys³⁷ PAL) | ″ |
| N-palmitate-GIP | Gault VA, Biochem J 2002; 367(Pt 3): 913-920 |
| N-fluorenyl methoxy carbonyl-GIP | ″ |
| (Ser²)-GIP | Gault VA, J Endocrinol 2003; 176(1): 133-141 |
| (Gly²)-GIP | ″ |
| GIP (mPEG) | Gault VA, Biochem Pharmacol 2008; 75(12)2325-2333 |
| GIP(1-30)-PEG | Salhanick AI, Bioorganic Med Chem Letters 2005; |
| | 15(18): 4114-4117 |
| Palm-GIP(1-30) | Salhanick AI, Bioorganic Med Chem Letters 2005; |
| | 15(18): 4114-4117 |
| GIP(1-30) | Salhanick AI, Bioorganic Med Chem Letters 2005; |
| | 15(18): 4114-4117 |
| Palm-GIP(1-30)-PEG | Salhanick AI, Bioorganic Med Chem Letters 2005; |
| | 15(18): 4114-4117 |
| Antagonists | |
| GIP(6-30)-NH₂ | Gelling RW, Reg Peptides 1997; 69(3): 151-154 |
| GIP(3-42) | Gault VA, J Endocrinol 2002; 175(2): 525-533 |
| (Pro³)-GIP | Gault VA, Diabetologia 2003; 46(2): 222-230 |
| (Hyp³)-GIP | O′Harte FPM, Am J Physiol Endocrinol Metab 2007; |
| | 292(6): E1674-E1682 |
| (Hyp³)-GIP-(Lys¹⁶ PAL) | O′Harte FPM, Am J Physiol Endocrinol Metab 2007; |
| | 292(6): E1674-E1682 |
| (Pro³)-GIP-[mPEG] | McClean PL, Br J Pharmacol 2008; 155(5): 690-701 |
| GIP(7-30)-NH₂ | Tseng CC, Am J Physiol 1999; 276(6): E1049-E1054 |

To estimate the suitability of a peptide receptor for in vivo tumor targeting, one needs detailed in vitro data on its expression in human tumors and human normal tissues. One critical prerequisite for a successful in vivo targeting is a high receptor expression in tumors, allowing a high tumoral radiotracer accumulation. Equally important is a low receptor expression in normal tissues surrounding tumors, at sites of tumor origin and of metastasis, since receptor targeted scintigraphy will detect tumors with adequate sensitivity only in case of a high tumor-to-background-signal-ratio. Moreover, knowledge of the distribution and putative functions of a peptide receptor in normal tissues is important in order to estimate the potential of side effects of a peptide therapy.

It has now been found that glucose-independent insulinotropic polypeptide (GIP) receptor plays a particular role in many tumor types.

A study to assess the GIP receptor protein expression was performed, firstly, in a broad spectrum of human tumors, and, secondly, in normal human tissues from common sites of tumor origin and metastasis using in vitro receptor autoradiography. This method has several advantages over other techniques: It identifies receptor binding sites which represent the in vivo target structures, it allows assessing the binding affinity of the receptor, and it permits correlation with morphology and quantification of the data. Moreover, the results of GIP receptor expression were compared with the somatostatin receptor and/or GLP-1 receptor status in the same tumors.

GIP Receptor Expression in Human Tumors

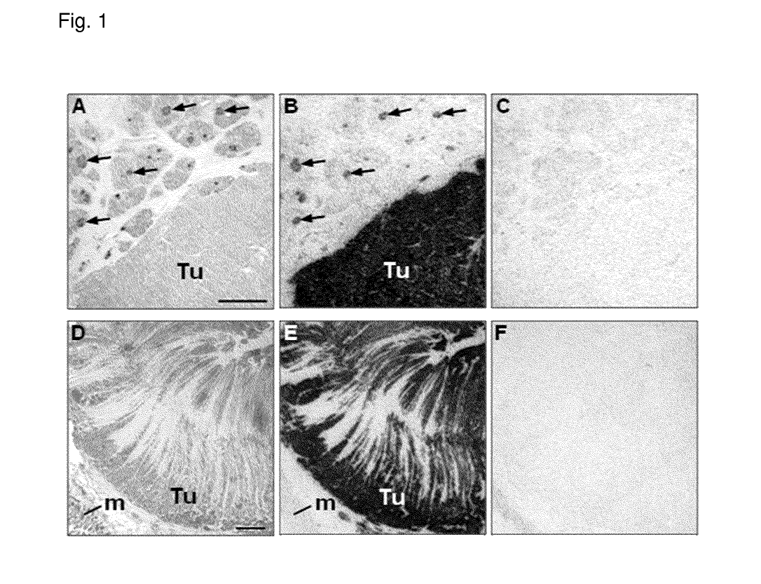

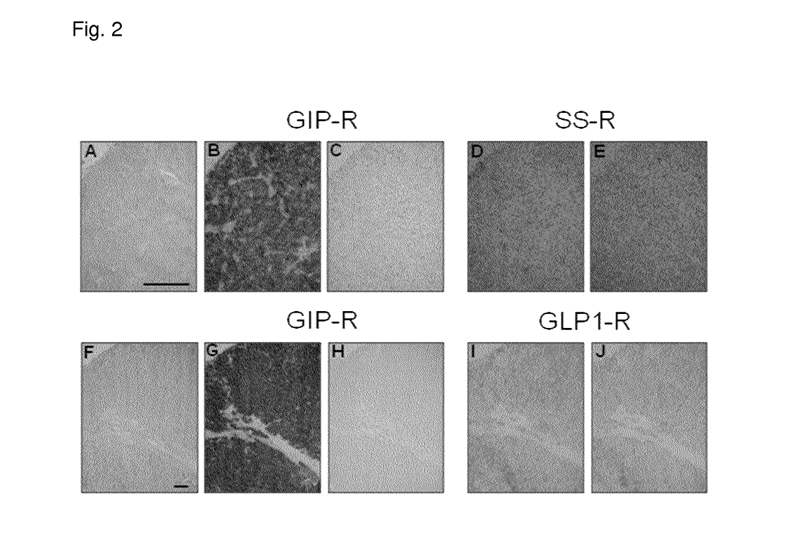

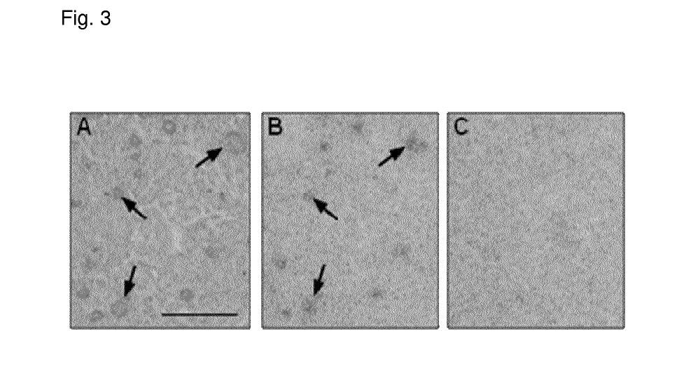

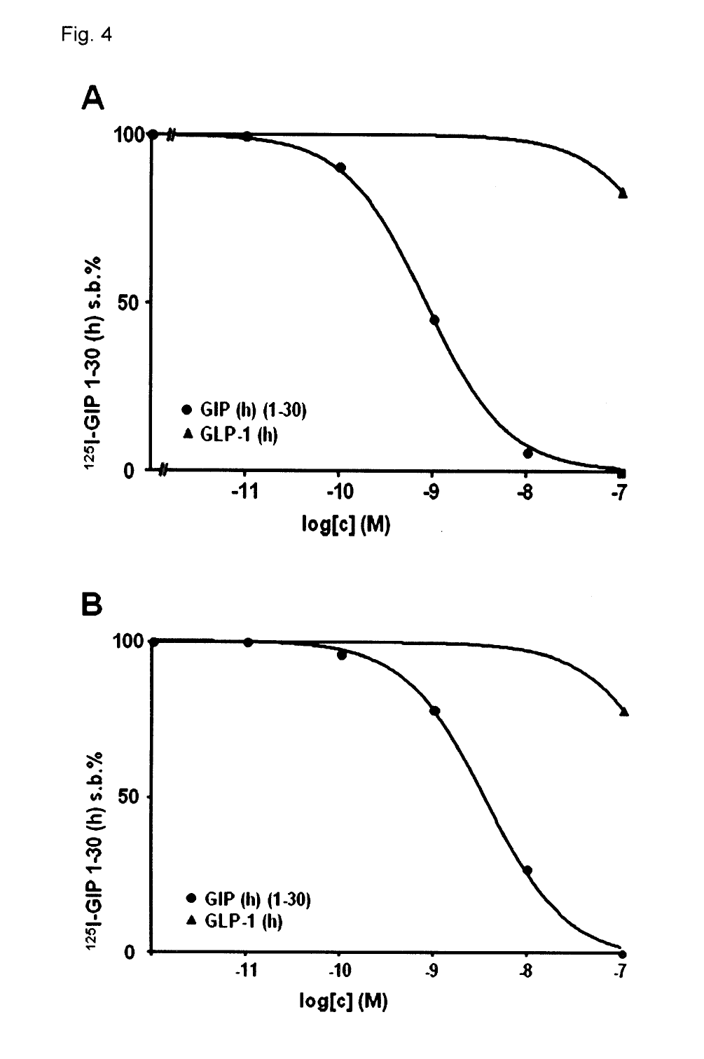

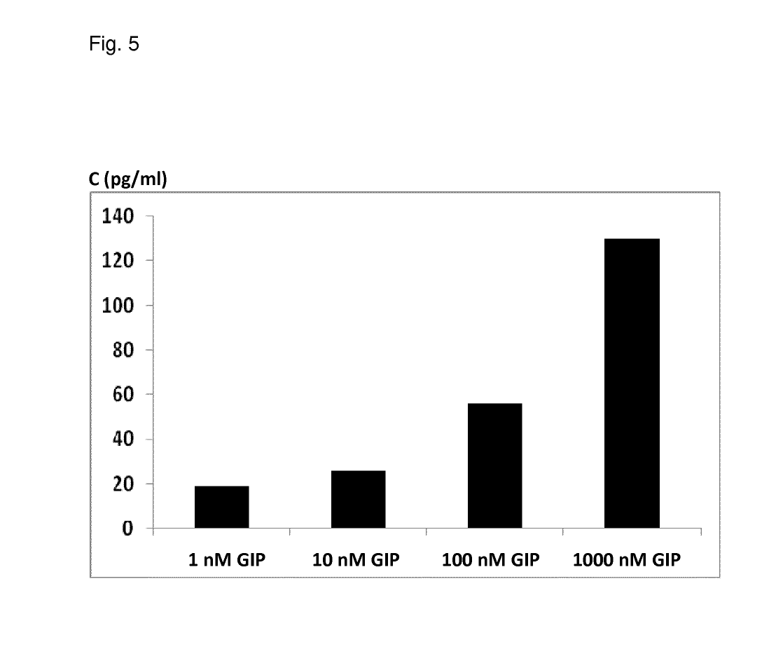

The GIP receptor expression was assessed in a broad spectrum of human gastrointestinal tumors. Table 2 summarizes the GIP receptor incidences and densities in these tumors. It shows that a high GIP receptor expression is found mainly in endocrine tumors. Of these tumors, functional pancreatic neuroendocrine tumors (NETs), including insulinomas, gastrinomas, glucagonomas and vipomas, as well as non-functional pancreatic NETs and ileal NETs, are especially noteworthy. Two representative examples are shown in FIG. 1. Remarkable is not only the high receptor density but also the homogeneous receptor distribution in both tumors. Of particular interest is the GIP receptor overexpression in the majority (88%) of somatostatin receptor-negative neuroendocrine tumors, which consist of gastrointestinal and bronchial and thyroid neuroendocrine tumors, and also in most GLP-1 receptor-negative malignant insulinomas (Table 2, FIG. 2). GIP receptors are also detected in most bronchial NETs. Conversely, among the epithelial cancers, GIP receptors are rarely found (Table 2). The highest incidence of GIP receptor expression, approximately 26%, is found in pancreatic tumors. This occasional GIP receptor expression is found both in pancreatic intraductal neoplasia and in invasive carcinoma; the only case with a high density of GIP receptors is a pancreatic intraductal neoplasia (FIG. 2). GIP receptors are not detected in colonic adenocarcinomas, hepatocellular carcinomas, GIST, gut lymphomas and rarely detected in gastric adenocarcinomas and cholangiocarcinomas (Table 2). Finally, a great majority of thyroid neuroendocrine tumors, the medullary thyroid carcinomas, express GIP receptors in high density (81% of cases) (Table 2).

GIP receptor incidence and density in human neuroendocrine tumors.
Comparison with SS-R in endocrine tumors

| | | | GIP receptor density** (number | | |
|---|---|---|---|---|---|
| | | | of cases per density category) | | |
| | | GIP receptor | low | moderate | high |
| Tumor type | | incidence* | <1000 dpm/mg | 1000-3000 dpm/mg | >3000 dpm/mg |
| Endocrine tumors | | | | | |
| Ileal NETs | | 18/21 | 0 | 3 | 15 |
| Pancreatic NETs | | | | | |
| Functional: | Insulinomas | 39/39 | 9 | 14 | 16 |
| | Gastrinomas | 9/9 | 0 | 0 | 9 |
| | Glucagonomas | 4/4 | 1 | 1 | 2 |
| | Vipomas | 4/4 | 1 | 0 | 3 |
| | Somatostatinomas | 1/2 | 0 | 0 | 1 |

| Non-functional: | | 15/15 | 0 | 4 | 11 |
|---|---|---|---|---|---|
| Carcinomas | | | | | |
| Colorectal adenocarcinomas | | 0/9 | | | |
| Gastric adenocarcinomas | | 1/12 | 1 | 0 | 0 |
| Pancreatic adenocarcinomas | | 5/19 | 4 | 0 | 1 |
| Cholangiocellular carcinomas | | 1/12 | 1 | 0 | 0 |
| Hepatocellular carcinomas | | 0/12 | | | |
| Stromal tumors | | | | | |
| GIST | | 0/12 | | | |
| Varia | | | | | |
| Bronchial NETs | | 22/24 | 8 | 8 | 6 |
| GI lymphomas | | 0/9 | | | |
| SS-R-neg. GEP tumors | | 39/44 | 7 | 18 | 14 |
| GLP-1R-neg. malign. insulinomas | | 4/4 | | | 4 |
| Medullary thyroid carcinomas | | 24/27 | 3578 ± 429 dpm/mg (n = 24) | | |
| | | | SS-R density (number of cases per | | |
| | | | density category) | | |
| | | SS-R | low | moderate | high |
| Tumor type | | incidence | <1000 dpm/mg | 1000-3000 dpm/mg | >3000 dpm/mg |
| Endocrine tumors | | | | | |
| Ileal NETs | | 18/21 | 1 | 4 | 13 |
| Pancreatic NETs | | | | | |
| Functional: | Insulinomas | 20/39 | 3 | 2 | 15 |
| | Gastrinomas | 8/9 | 0 | 0 | 8 |
| | Glucagonomas | 3/4 | 0 | 1 | 2 |
| | Vipomas | 4/4 | 0 | 0 | 4 |
| | Somatostatinomas | 0/2 | 0 | 0 | 0 |

| | | | | |
|---|---|---|---|---|
| Non-functional: | 13/15 | 0 | 0 | 13 |
| Carcinomas | | | | |
| Colorectal adenocarcinomas | | | | |
| Gastric adenocarcinomas | | | | |
| Pancreatic adenocarcinomas | | | | |
| Cholangiocellular carcinomas | | | | |
| Hepatocellular carcinomas | | | | |
| Stromal tumors | | | | |
| GIST | | | | |
| Varia | | | | |
| Bronchial NETs | 12/24 | 0 | 2 | 10 |
| GI lymphomas | | | | |
| SS-R-neg. GEP tumors | 0/44 | | | |
| GLP-1R-neg. malign. insulinomas | 4/4 | | | 4 |

| | |
|---|---|
| Medullary thyroid carcinomas | NA |

*Values in parentheses are percentages
**Mean ± SEM of receptor-positive cases (dpm/mg tissue)
NA, not assessed

GIP Receptor Expression in Non-Neoplastic Human Tissues

GIP receptors were also investigated in a wide variety of non-neoplastic human tissues of the GI tract. They are found only in few specific organs and in specific tissue compartments. The results are summarized in Table 3. Of particular relevance, because physiologically functional, are the GIP receptors in pancreatic islets. They are detected in comparable amounts in islets of donor pancreas and of pancreas from NET patients (FIG. 3). They are also expressed in islets of pancreas from chronic pancreatitis and pancreatic carcinomas patients, however, with a trend of higher receptor density (Table 3). Of note, the acini in the pancreas do not express GIP receptors in any of the patient categories (FIG. 3, Table 3). No GLP-1 receptors are found in the following tissues: stomach, duodenum, ileum, colon, liver, and rarely in gall bladder, lymph nodes or lung.

TABLE 3
GIP receptor density in receptor-positive human non-neoplastic
gastrointestinal tissues

| | Tissue | Organ | GIP receptor | GIP receptor |
|---|---|---|---|---|
| Organ | compartment | condition | incidence | density* |
| Pancreas | Islets | donor | 4/4 | 563 ± 110 |
| | | NET | 18/18 | 801 ± 63 |
| | | pancreas Ca | 15/15 | 1514 ± 123 |
| | | pancreatitis | 7/7 | 1288 ± 487 |
| | Acini | donor | 0/4 | |
| | | NET | 0/18 | |
| | | pancreas Ca | 0/15 | |
| | | pancreatitis | 0/11 | |
| Stomach | | | 0/8 | |
| Duodenum | | | 0/6 | |
| Ileum | | | 0/9 | |
| Colon | | | 0/9 | |
| Liver | | | 0/12 | |
| Gall bladder | | | 1/6 | |
| Lymph nodes | | | 1/15 | |
| Lung | | | 2/11 | |

*Mean ± SEM of receptor-positive cases (dpm/mg tissue)

Pharmacological Characterization of GIP Receptors

To prove that the radioligand ¹²⁵I-GIP(1-30) was specifically bound by GIP receptors in the autoradiography experiments, pharmacological displacement experiments were performed using ¹²⁵I-GIP(1-30) in competition with increasing concentrations of cold GIP(1-30), GIP, the GLP-1 receptor-selective agonist GLP-1, the GLP-2 receptor selective agonist GLP-2, or the glucagon receptor selective agonist glucagon(1-29). Representative results for a tumor and normal pancreatic islets are shown in FIG. 4. In all examples, ¹²⁵I-GIP(1-30) is displaced by GIP(1-30) and GIP with high affinity in the nanomolar concentration range, whereas it is not displaced by GLP-1, GLP-2 and glucagon(1-29). This rank order of potencies provides strong pharmacological evidence that GIP receptors are specifically identified.

The present report on GIP receptor protein expression in a large spectrum of human neuroendocrine tumors and adjacent normal human tissues represents a significant extension of current knowledge on the tumoral and physiological expression of gut hormone receptors in humans. It shows, for the first time, that GIP receptors are massively overexpressed in specific neuroendocrine tumors (NETs), whereas they are virtually absent in gastrointestinal (GI) carcinomas, sarcomas and lymphomas. An impressive GIP receptor expression is found in functional pancreatic NETs, such as insulinomas and gastrinomas, as well as non-functional pancreatic NETs and ileal NETs, but also in most bronchial NETs and in most medullary thyroid carcinomas, characterized by both a very high receptor incidence and density. The high receptor content in neuroendocrine tumors contrasts with the low physiological GIP receptor expression in corresponding healthy human tissues, with only few gastrointestinal tissues showing measurable receptor amounts, primarily the islets of the pancreas, where GIP has its main physiological action.

It has previously been shown that a large percentage of endocrine gastroenteropancreatic tumors express receptors for another gut peptide, somatostatin. The somatostatin receptors have been reported in the majority of well-differentiated pancreatic NETs, in virtually all gastrinomas, however, to a much lesser extend in insulinomas and undifferentiated NETs. They have been found more rarely in bronchial than in gut NETs. These receptors have been the basis for in vivo somatostatin receptor targeting of neuroendocrine tumors; the use of this technique has considerably changed and improved the clinical strategy in the respective patients. Unfortunately, patients bearing tumors belonging to the minority of neuroendocrine tumors that are lacking somatostatin receptors cannot benefit from imaging or targeted radiotherapy. A most significant result of the comparison of GIP receptor expression with somatostatin receptor expression in all neuroendocrine tumors is therefore that GIP receptor incidence compared advantageously with the incidence of somatostatin receptor expression in this group of gastroenteropancreatic endocrine tumors. The fact that somatostatin receptor-negative tumors retain the GIP receptors is of clinical significance. GIP receptor imaging as described herein replaces or complements somatostatin receptor imaging in these tumors. Particularly interesting cases are the insulinomas; GIP receptors are not only expressed in all benign insulinomas, including the somatostatin receptor-negative ones, but also in malignant insulinomas, known to often lack another gut peptide receptor, the GLP-1 receptor. As such, GIP receptor imaging is a universal marker for neuroendocrine tumors. The GIP receptor containing neuroendocrine tumors represent prospective candidates for an in vivo targeting for imaging and therapy analogous to the somatostatin receptor targeting.

The generally low physiological GLP-1 receptor expression in humans, in sites of the GI tract primaries as well as in common sites of metastases such as lymph nodes, liver, or lung represents a favorable circumstance for a GIP receptor tumor imaging. Indeed, a high tumor-to-background-ratio (background: non neoplastic surrounding tissues) is an essential prerequisite for a sensitive and specific tumor detection with receptor targeted scintigraphy. In addition, the results of the present invention add valuable information to the receptor status in normal human tissues. GIP receptors were detected in measurable amounts only in selected normal gastrointestinal tissue compartments, such as islets of the pancreas. Similar amounts of GIP receptors are expressed in the pancreatic islets from donor pancreas or NET-bearing pancreas. Interestingly, but difficult to explain, is the trend of higher GIP receptor expression in pancreatic islets from evidently altered pancreas, such as pancreatitis or pancreatic adenocarcinomas. Furthermore, of particular importance is the observation that the pancreatic acini completely lack GIP receptors in all tested pancreatic conditions. This is at difference from the GLP-1 receptors which have been shown to be expressed both in islets and acini. These results indicate that the GIP receptor is likewise a target for β-cell mass imaging, since it targets only the β-cell in the pancreas.

The high GIP receptor expression in specific endocrine tumors and low expression in normal tissues represent the molecular basis for an in vivo neuroendocrine tumor targeting for diagnostic and therapeutic purposes. While this is particularly true for those tumors expressing no other gut peptide receptor than GIP receptors, the frequent concomitant expression of GIP receptors with somatostatin receptors and even GLP-1 receptors in many NETs suggest the possibility of multiple receptor targeting of the respective tumors; injections of a cocktail of established radiolabeled somatostatin analogs (Reubi J C and Maecke H R, J Nucl Med 2008; 49:1735-8) and GLP-1 analogs

(Christ E, Wild D, Forrer F et al, J Clin Endocrinol Metab 2009; 94:4398-4405; Wild D, Mäcke H, Christ E et al, N Engl J Med 2008; 359:766-8), together with GIP analogs provides extremely potent tumor imaging and targeted radiotherapy.

Stable and adequately labeled GIP analogs suitable for nuclear medicine applications considered are, for example, those GIP analogs listed in Table 1, carrying radionuclides, preferably complexed by chelators. The chelators should preferably not be located at the N-terminal end of GIP, since this end binds to the GIP receptor pocket.

Chelators considered to be attached to the GIP analogs are the usual radionuclide chelators, preferably attached to the C-terminal end of the GIP analogs, for example DOTA- and DTPA-based chelators, NOTA-based chelators, chelating carbonyl compounds, 2-hydrazino nicotinamide (HYNIC), N₄-chelators, desferrioxamin, and NₓS_{y}-chelators, all optionally complexed with a radioisotope. Tyrosine (Tyr) may be attached to the GIP analog for halogenation, reaction with a fluorescent dye, or with biotin, to be used for non-radioactive tumour imaging. Cpa (4-chloro-L-phenylalainine) may also serve as a precursor for tritiation. For example, a chelator, such as DTPA (diethyleneamine-N,N,N′,N″,N″-pentaacetic acid), DOTA (1,4,7,10-tetraazacyclododecane-1,4,7,10-tetraacetic acid), NODAGA ((1-(1,3-dicarboxypropyl)-1,4,7-triazacyclononane,-4,7-diacetic acid), HYNIC (6-(2-carboxyhydrazinyl)pyridine-3-carboxylic acid) and P2S2-COOH (Dithio-diphosphine based bifunctional chelating agents) may be attached. Preferred chelators include p-NH₂-Bz-DOTA (2-p-aminobenzyl-1,4,7,10-tetraazacyclododecane-1,4,7,10-tetraacetic acid), and DOTA-p-NH₂-anilide [1,4,7,10-tetraazacyclododecane-1,4,7,10-tetraacetic acid mono(p-aminoanilide)]. Alternatively, a chelating agent may be covalently linked to the N-terminal end via a suitable linker (L), if desired. Suitable linkers L include tyrosine, lysine, diaminobutyric acid, diaminopropionic acid, polyethylene glycol, fatty acids and their derivatives, β-alanine, 5-aminovaleric acid, sarcosine, and glucuronic acid. When Tyr appears at the N-terminus, it may be radioiodinated or otherwise labeled. Acyl groups having not more than about 20 amino acids may also be present at the N-terminus, and the N-terminal residue may also be acylated, if desired, with a bulky moiety without loss of selectivity.

Radionuclides considered effective for scintigraphy or for combating or controlling tumors are selected from the group consisting of ¹⁸⁶Re, ¹⁸⁸Re, ¹¹¹In, ¹¹³In, ⁷¹As, ⁹⁰Y, ⁶⁷Cu, ^{99m}Tc, ¹⁶⁹Er, ¹²¹Sn, ¹²⁷Te, ¹⁴²Pr, ¹⁴³Pr, ⁶⁶Ga, ⁶⁷Ga, ⁶⁸Ga, ⁷²Ga, ¹²⁷Te, ¹⁹⁵Pt, ²¹¹At, ¹⁹⁸Au, ¹⁹⁹Au, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁵Yb, ¹⁷⁷Lu, ¹⁰⁵Rh, ¹¹⁴Ag, ¹²⁴I and ¹³¹I. Radionuclides particularly suitable for tumor imaging are, for example, gamma emitters, such as ^{99m}Tc, ¹⁶¹Tb, ⁶⁷Ga, ⁶⁸Ga, ¹¹¹In, ¹⁷⁷Lu, ¹²³I or ¹²⁵I, and beta emitters such as ⁹⁰Y and ¹⁷⁷Lu, and positron emitters such as ¹⁸F.

Other substituents considered for GIP and GIP analogs to be used in tumor therapy are standard anti-neoplastic medicaments, for example, antimetabolites such as 5-fluorouracil or gemcitabine HCl, alkylating agents such as oxaliplatin, dacarbazin, cyclophosphamide or carboplatin, cell-cycle inhibitor such as vinorelbine, vinblastine or docetaxel, DNA breaker (topo-isomerase inhibitor, intercalator, strand breaker) such as doxorubicin HCl, bleomycin, irinotecan, etoposide phosphate or topotecan HCl, and related compounds used in tumor therapy.

The invention relates to a method of imaging pancreatic β-cells, endocrine gastroenteropancreatic tumors and bronchial and thyroid neuroendocrine tumors, and a method of treating endocrine gastroenteropancreatic tumors and bronchial and thyroid neuroendocrine tumors by targeting of glucose-independent insulinotropic polypeptide receptors (GIP receptors).

In particular, the invention relates to a method of imaging pancreatic β-cells, endocrine gastroenteropancreatic tumors and bronchial and thyroid neuroendocrine tumors comprising administering glucose-independent insulinotropic polypeptide (GIP) or a GIP analog, each carrying a radionuclide, optionally complexed through a chelator, or other substituent useful for imaging. Likewise the invention relates to glucose-independent insulinotropic polypeptide (GIP) or a GIP analog, each carrying a radionuclide, optionally complexed through a chelator, or other imaging substituent for use in imaging pancreatic β-cells, endocrine gastroenteropancreatic tumors and bronchial and thyroid neuroendocrine tumors. Particular radionuclides considered are those listed above as suitable for tumor imaging. In GIP or GIP analogs carrying ¹²³I or ¹²⁶I, these radionuclides may be directly attached to one of the amino acids of GIP. Other radionuclides are complexed through a chelator, such as those chelators mentioned above, in particular those mentioned as preferred.

Likewise the invention relates to a method of treating endocrine gastroenteropancreatic tumors and bronchial and thyroid neuroendocrine tumors comprising administering a therapeutically effective amount of glucose-independent insulinotropic polypeptide (GIP) or a GIP analog, each carrying a radionuclide, optionally complexed through a chelator, or other substituent useful for tumor treatment, to a patient in need thereof. Likewise the invention relates to glucose-independent insulinotropic polypeptide (GIP) or a GIP analog, each carrying a radionuclide, optionally complexed through a chelator, or other substituent useful for tumor treatment, for use in treating endocrine gastroenteropancreatic tumors and bronchial and thyroid neuroendocrine tumors. Particular radionuclides considered are those listed above. Particular substituents useful for tumor treatment are likewise those listed above.

GIP analogs considered are, in particular, those listed in Table 1.

Furthermore, the invention relates to a method of treatment of endocrine gastroenteropancreatic tumors and bronchial and thyroid neuroendocrine tumors comprising administering combinations of GIP or a GIP analog, each carrying a radionuclide, optionally complexed through a chelator, or other substituent useful for tumor treatment, together with suitable GLP-1 agonists and/or somatostatin analogs, each carrying a radionuclide. Likewise the invention relates to such combinations of GIP or a GIP analog, each carrying a radionuclide, optionally complexed through a chelator, or other substituent useful for tumor treatment, together with suitable GLP-1 agonists and/or somatostatin analogs, each carrying a radionuclide, for use in the treatment of gastroenteropancreatic tumors and bronchial and thyroid neuroendocrine tumors.

GLP-1 agonists considered are, for example, synthetic GLP-1 analogs such as exenatide, liraglutide or taspoglutide, and also GLP-1 analogs such as exendin-3 and exendin-4, carrying radionuclides, such as those listed above.

Somatostatin analogs considered are those known in the art, for example octreotide, Nal³-octreotide (Nal=1-naphthylalanine), benzothienylalanine³-octreotide, Tyr³-octreotide, Tyr³, Thr⁸-octeotride, des-AA^{1,2,4,5,12,13}[D-2Nal⁸]-somatostatin-14 and others, preferably carrying radionuclides, such as those listed above.

Endocrine gastroenteropancreatic tumors considered are ileal neuroendocrine tumors and pancreatic neuroendocrine tumors, such as insulinomas, gastrinomas, glucagonomas, vipomas, and non-functional pancreatic neuroendocrine tumors. Included in “endocrine gastroenteropancreatic tumors and bronchial and thyroid neuroendocrine tumors” are also metastases derived from such tumors and appearing in other organs.

It has further been found that GIP stimulates calcitonin release in TT cells; calcitonin itself is known to stimulate proliferation. TT cells are human medullary thyroid cancer cells, and are therefore an established representative of human neuroendocrine tumors. Because GIP is known to stimulate cell proliferation in normal pancreatic β-cells, hippocampus and tumoral MC-26 tissues (Prabakaran D. et al., Regul Peptides 2010, 163:74-80), non-radioactive GIP analogs, in particular GIP receptor antagonists, will be able to inhibit cell proliferation in tumors expressing GIP receptors and therefore be useful for long-term therapy in patients bearing these tumors.

The invention therefore relates to a method of treating endocrine gastroenteropancreatic tumors and bronchial and thyroid neuroendocrine tumors comprising administering a therapeutically effective amount of a GIP analog, preferably a GIP-R antagonist, to a patient in need thereof, and likewise to a GIP-R antagonist for use in treating endocrine gastroenteropancreatic tumors and bronchial and thyroid neuroendocrine tumors. Particular GIP receptor antagonists considered are GIP(6-30)-NH₂, GIP(3-42), (Pro³)-GIP, (Hyp³)-GIP, (Hyp³)-GIP-(Lys¹⁶ PAL), (Pro³)-GIP-[mPEG], and GIP(7-30)-NH₂.

### EXAMPLES

Tissues

Fresh frozen samples of tumor tissues obtained from surgical resection specimens and characterized previously for other peptide receptors (Reubi J C and Waser B, Eur J Nucl Med 2003; 30:781-793) were used. Furthermore, non-neoplastic human tissues resected together with the tumor or adjacent to the tumor, were also included. The study conformed to the ethical guidelines of the Institute of Pathology, University of Bern, and was reviewed by the Institutional Review Board.

In Vitro GIP Receptor Autoradiography

The in vitro GIP receptor autoradiography was carried out as described previously for the GLP-1 receptor (Reubi J C and Waser B, Eur J Nucl Med 2003; 30:781-793; Korner M, Stockli M, Waser B et al, J Nucl Med 2007; 48:736-43). The peptide analog used as radioligand was human GIP(1-30). It was radiolabeled by the lactoperoxidase method and purified by HPLC (Anawa, Wangen, Switzerland). The two lodo-Tyrosine analogues (peak 1 iodinated at Tyr¹ and peak 2 iodinated at Tyr¹⁰) were analyzed by LC-ESI-MS-MS (R. Brunisholz, Functional Genomics Center Zürich, ETHZ). The peak (2′000 Ci/mmol) representing ¹²⁵I-[Tyr¹⁰]-GIP(1-30) was used in all experiments. Twenty micrometer thick frozen tissue sections were incubated for 2 hours at room temperature in the incubation solution containing 170 mM Tris-HCl buffer (pH 8.2), 1% bovine serum albumin (BSA), 40 μg/ml bacitracin, 10 mM MgCl₂, and 20,000 cpm/100 μl ¹²⁵I-GIP(1-30). Non-specific binding was determined by incubating tissue sections in the incubation solution containing additionally 100 nM unlabeled human GIP (Bachem, Bubendorf, Switzerland) which at this concentration completely and specifically displaces ¹²⁵I-GIP(1-30) binding at the receptors. Further pharmacological displacement experiments were performed in order to differentiate GIP receptors from other members of the glucagon receptor family. For this purpose, serial tissue sections were incubated with ¹²⁵I-GIP(1-30) together with increasing concentrations of one of the following analogues: human GIP, the GLP-1 receptor-selective analogue GLP-1 (Bachem), the GLP-2 receptor-selective analogue GLP-2 (Bachem) or the glucagon receptor-selective analogue glucagon(1-29) (Bachem). After incubation, the slides were washed five times in ice-cold Tris-HCl buffer (170 mM; pH 8.2) containing 0.25% BSA and twice in ice-cold Tris-HCl buffer without BSA. The slides were dried for 15 minutes under a stream of cold air and then exposed to Kodak films Biomax MR® for seven days at 4° C. The signals on the films were analyzed in correlation with morphology using corresponding H&E stained tissue slides. The receptor density was quantitatively assessed using tissue standards for iodinated compounds (Amersham, Aylesbury, UK) and a computer-assisted image processing system (Analysis Imaging System, Interfocus, Mering, Germany).

In Vitro GLP-1 and Somatostatin Receptor Autoradiography

GLP-1 receptor expression was evaluated in vitro by GLP-1 receptor autoradiography as previously reported using ¹²⁵I-GLP-1 (7-36)amide (74 Bq/mmol; Anawa, Wangen, Switzerland) as radioligand in sections of patients' tumor samples (Korner M, Stockli M, Waser B et al, J Nucl Med 2007; 48:736-43). The in vitro autoradiography of somatostatin receptor expression was performed in consecutive sections of the same tumor using the sst₂-prefering radioligand ¹²⁵I-[Tyr³]-Octreotide as described in Reubi J C, Kvols L K, Waser B et al., Cancer Res. 1990; 50:5969-5977.

Action of GIP on Calcitonin Levels

TT cells (derived from a human medullary thyroid carcinoma; ATCC Number: CRL-1803) were plated in 24-well plates (100′000 cells per well) and cultured for 48 hours in growth medium (nutrient mixture F12 Ham Kaighn's modification containing L-glutamine and supplemented with 10% fetal bovine serum) at 37° C. and 5% CO₂. Subsequently the growth medium was replaced by the serum-free challenge medium (RPMI 1640, 10 mM HEPES and GlutaMax-I) and the cells were incubated for further 48 hours at 37° C. and 5% CO₂. Then the challenge medium was removed and the cells were stimulated by the addition of challenge medium containing different concentrations of GIP in the range from 1 nM up to 1 μM. As negative control cells were treated with challenge medium containing vehicle alone. The cells were incubated for 3 hours at 37° C. and 5% CO₂. After GIP-stimulation the supernatant was collected and the calcitonin level was determined using the Calcitonin-Kit 100T (Siemens Healthcare; Product-No. 06601463).

β-Cell Imaging with GIP Radioligands

Sprague-Dawley rats were administrated ¹¹¹In-DOTA-GIP(1-30) i.v. with or without unlabeled GIP(1-30) to determine binding specificity. Animals were euthanized and the pancreas was extracted, immediately frozen, and sectioned. The sections were apposed to radiosensitive films, scanned, and immunostained for insulin. Correlation of the autoradiographic and immunohistochemical images reveals that GIP binding was restricted to islet cells, indicatin specific β-cell imaging.

1. A method of imaging pancreatic β-cells, endocrine gastroenteropancreatic tumors and bronchial and thyroid neuroendocrine tumors comprising administering glucose-independent insulinotropic polypeptide (GIP) or a GIP analog, each carrying a radionuclide, optionally complexed through a chelator, or other substituent useful for imaging.

2. A method of imaging endocrine gastroenteropancreatic tumors and bronchial and thyroid neuroendocrine tumors according to claim 1.

3. A method of imaging pancreatic β-cells according to claim 1.

4. Glucose-independent insulinotropic polypeptide (GIP) or a GIP analog, each carrying a radionuclide, optionally complexed through a chelator, or other imaging substituent, for use in imaging pancreatic β-cells, endocrine gastroenteropancreatic tumors and bronchial and thyroid neuroendocrine tumors.

5. A method of treating endocrine gastroenteropancreatic tumors and bronchial and thyroid neuroendocrine tumors comprising administering a therapeutically effective amount of glucose-independent insulinotropic polypeptide (GIP) or a GIP analog, each carrying a radionuclide, optionally complexed through a chelator, or other substituent useful for tumor treatment, to a patient in need thereof.

6. Glucose-independent insulinotropic polypeptide (GIP) or a GIP analog, each carrying a radionuclide, optionally complexed through a chelator, or other substituent useful for tumor treatment, for use in treating endocrine gastroenteropancreatic tumors and bronchial and thyroid neuroendocrine tumors.

7. A method of treating endocrine gastroenteropancreatic tumors and bronchial and thyroid neuroendocrine tumors comprising administering a therapeutically effective amount of a glucose-independent insulinotropic polypeptide receptor (GIP-R) antagonist, to a patient in need thereof.

8. A glucose-independent insulinotropic polypeptide receptor (GIP-R) antagonist for use in treating endocrine gastroenteropancreatic tumors and bronchial and thyroid neuroendocrine tumors.

9. The GIP-R antagonist for use in treating endocrine gastroenteropancreatic tumors and bronchial and thyroid neuroendocrine tumors according to claim 8 selected from GIP(6-30)-NH₂, GIP(3-42), (Pro³)-GIP, (Hyp³)-GIP, (Hyp³)-GIP-(Lys¹⁶ PAL), (Pro³)-GIP-[mPEG], and GIP(7-30)-NH₂.

10. GIP or the GIP analog according to claim 4 comprising a radionuclide.

11. GIP or the GIP analog according to claim 10 comprising a radionuclide selected from ^{99m}Tc, ¹⁶¹Tb, ⁶⁷Ga, ¹¹¹In, ¹⁷⁷Lu, ¹²³I or ¹²⁵I.

12. GIP or the GIP analog according to claim 10 comprising a radionuclide selected from ¹⁸⁶Re, ¹⁸⁸Re, ¹¹¹In, ^{113m}In, ⁷¹As, ⁹⁰Y, ⁶⁷Cu, ^{99m}Tc, ¹⁶⁹Er, ¹²¹Sn, ¹²⁷Te, ¹⁴²Pr, ¹⁴³Pr, ⁶⁶Ga, ⁶⁷Ga, ⁶⁸Ga, ⁷²Ga, ¹²⁷Te, ¹⁹⁵Pt, ²¹¹At, ¹⁹⁸Au, ¹⁹⁹Au, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, 175 _{Yb,} ¹⁷⁷Lu, ¹⁰⁵Rh, ¹¹⁴Ag, ¹²⁴I and ¹³¹I.

13. GIP or the GIP analog according to claim 10 wherein the radionuclide is complexed through a chelator.

14. GIP or the GIP analog according to claim 13 wherein the chelator is selected from DOTA- and DTPA-based chelators, NOTA-based chelators, NODAGA-based chelators, chelating carbonyl compounds, 2-hydrazino nicotinamide type chelators, N₄-chelators, desferrioxamin, and NₓS_{y}-chelators.

15. GIP or the GIP analog according to claim 6 substituted with an anti-neoplastic medicament.

16. GIP or the GIP analog according to claim 15 substituted with an antimetabolite, alkylating agent, cell-cycle inhibitor, or DNA breaker.

17. The GIP analog according to claim 4anyone of claims 4 selected from N-Ac GIP (Lys³⁷ PAL), D-Ala²-GIP, D-Ala²-GIP(1-30), GIP (Lys¹⁶ PAL), GIP (Lys³⁷ PAL), N-Ac GIP, N-Ac GIP (Lys³⁷ PAL), N-palmitate-GIP, N-fluorenylmethoxycarbonyl-GIP, (Ser²)-GIP, (Gly²)-GIP, GIP (mPEG), GIP(1-30)-PEG, Palm-GIP(1-30)-PEG, GIP(6-30)-NH₂, GIP(3-42), (Pro³)-GIP, (Hyp³)-GIP, (Hyp³)-GIP-(Lys¹⁶ PAL), (Pro³)-GIP-[mPEG], and GIP(7-30)-NH₂.

18. A combination of GIP or a GIP analog, each carrying a radionuclide, optionally complexed through a chelator, or other substituent useful for tumor treatment, together with a GLP-1 agonist and/or somatostatin analog, each carrying a radionuclide, for use in the treatment of gastroenteropancreatic tumors and bronchial and thyroid neuroendocrine tumors.

19. A combination of a GIP-R antagonist, together with a GLP-1 agonist and/or somatostatin analog, each carrying a radionuclide, for use in the treatment of gastroenteropancreatic tumors and bronchial and thyroid neuroendocrine tumors.

20. The method according to claim 1 wherein the gastroenteropancreatic tumor is selected from ileal neuroendocrine tumors, insulinomas, gastrinomas, glucagonomas, vipomas, and non-functional pancreatic neuroendocrine tumors.

21. GIP or the GIP analog according to claim 4 for use in imaging or treating a gastroenteropancreatic tumor selected from ileal neuroendocrine tumors, insulinomas, gastrinomas, glucagonomas, vipomas, and non-functional pancreatic neuroendocrine tumors.

22. The GIP-R antagonist for use in treating a gastroenteropancreatic tumor selected from ileal neuroendocrine tumors, insulinomas, gastrinomas, glucagonomas, vipomas, and non-functional pancreatic neuroendocrine tumors.

23. GIP or the GIP analog according to claim 6 comprising a radionuclide.

24. GIP or the GIP analog according to claim 23 comprising a radionuclide selected from ^{99m}Tc, ¹⁶¹Tb, ⁶⁷Ga, ¹¹¹In, ¹⁷⁷Lu, ¹²³I or ¹²⁵I.

25. GIP or the GIP analog according to claim 23 comprising a radionuclide selected from ¹⁸⁶Re, ¹⁸⁸Re, ¹¹¹In, ^{113m}In, 71As, 90Y, ⁶⁷Cu, ^{99m}Tc, ¹⁶⁹Er, ¹²¹Sn, ¹²⁷Te, ¹⁴²Pr, ¹⁴³Pr, ⁶⁶Ga, ⁶⁷Ga, ⁶⁸Ga, ⁷²Ga, ¹²⁷Te, ¹⁹⁵Pt, ²¹¹At, ¹⁹⁸Au, ¹⁹⁹Au, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁶⁶Ho, ¹⁷²Tm, ¹⁶⁹Yb, ¹⁷⁵Yb, ¹⁷⁷Lu, ¹⁰⁵Rh, ¹¹⁴Ag, ¹²⁴I and ¹³¹I.

26. GIP or the GIP analog according to claim 23 wherein the radionuclide is complexed through a chelator.

27. GIP or the GIP analog according to claim 26 wherein the chelator is selected from DOTA- and DTPA-based chelators, NOTA-based chelators, NODAGA-based chelators, chelating carbonyl compounds, 2-hydrazino nicotinamide type chelators, N₄-chelators, desferrioxamin, and NₓS_{y}-chelators.

28. The GIP analog according to claim 6 selected from N-Ac GIP (Lys³⁷ PAL), D-Ala²-GIP, D-Ala²-GIP(1-30), GIP (Lys¹⁶ PAL), GIP (Lys³⁷ PAL), N-Ac GIP, N-Ac GIP (Lys³⁷ PAL), N-palmitate-GIP, N-fluorenylmethoxycarbonyl-GIP, (Ser²)-GIP, (Gly²)-GIP, GIP (mPEG), GIP(1-30)-PEG, Palm-GIP(1-30)-PEG, GIP(6-30)-NH₂, GIP(3-42), (Pro³)-GIP, (Hyp³)-GIP, (Hyp³)-GIP-(Lys¹⁶ PAL), (Pro³)-GIP-[mPEG], and GIP(7-30)-NH₂.

29. The GIP analog according to claim 10 selected from N-Ac GIP (Lys³⁷ PAL), D-Ala²-GIP, D-Ala²-GIP(1-30), GIP (Lys¹⁶ PAL), GIP (Lys³⁷ PAL), N-Ac GIP, N-Ac GIP (Lys³⁷ PAL), N-palmitate-GIP, N-fluorenylmethoxycarbonyl-GIP, (Ser²)-GIP, (Gly²)-GIP, GIP (mPEG), GIP(1-30)-PEG, Palm-GIP(1-30)-PEG, GIP(6-30)-NH₂, GIP(3-42), (Pro³)-GIP, (Hyp³)-GIP, (Hyp³)-GIP-(Lys¹⁶ PAL), (Pro³)-GIP-[mPEG], and GIP(7-30)-NH₂.

30. The method according to claim 5 wherein the gastroenteropancreatic tumor is selected from ileal neuroendocrine tumors, insulinomas, gastrinomas, glucagonomas, vipomas, and non-functional pancreatic neuroendocrine tumors.

31. The method according to claim 7 wherein the gastroenteropancreatic tumor is selected from ileal neuroendocrine tumors, insulinomas, gastrinomas, glucagonomas, vipomas, and non-functional pancreatic neuroendocrine tumors.

32. GIP or the GIP analog according to claim 6 for use in imaging or treating a gastroenteropancreatic tumor selected from ileal neuroendocrine tumors, insulinomas, gastrinomas, glucagonomas, vipomas, and non-functional pancreatic neuroendocrine tumors.

33. GIP or the GIP analog according to claim 10 for use in imaging or treating a gastroenteropancreatic tumor selected from ileal neuroendocrine tumors, insulinomas, gastrinomas, glucagonomas, vipomas, and non-functional pancreatic neuroendocrine tumors.
